(19) United States
(12) Patent Application Publication
(10) Pub. No.: US 2009/0162805 A1
(43) Pub. Date: Jun. 25, 2009

(54) SYSTEM FOR MAKING CONICAL BONES IN ORGANIC TISSUES, AND A METHOD THEREFOR

(76) Inventor: Norberto Berna, Rome (IT)

Correspondence Address: YOUNG & THOMPSON, 209 Madison Street, Suite 500, ALEXANDRIA, VA, 22314, US

(21) Appl. No.: 12/294,250

(22) PCT Filed: Mar. 23, 2007
(86) PCT No.: PCT/IT2007/000215

§ 371 Date: Oct. 21, 2008

(30) Foreign Application Priority Data
Mar. 24, 2006 (IT) RM2006A000167

(51) Int. Cl. A61C 3/02; A61B 17/00

(52) U.S. Cl. 433/3; 606/79; 433/215

(57) ABSTRACT

A method and a system including templates (1,5,7) for the making of non cylindrical bores in organic tissues or the like and with a surgical tool is provided, wherein each template (1,5,7) includes an annular member (2) having a substantially elliptical or “O” elongated shape, and an elongated member (3) to be inserted in the tissue and having a predetermined length which defines the depth of the bore in the tissue, the elongated member being integrally connected at an end thereof to the annular member (2) and at the opposite end being shaped with a concave end part (4).

### FIELD OF THE INVENTION

The invention relates to a system for making conical bores in organic tissues, and a method therefor by means of surgical tools such as a mechanical mill, a supersonic mill, a laser or the like.

### STATE OF THE ART

To now, in the biomedical practice it is know the need to make bores in organic tissues for the sake of implanting prosthesis or the like. In particular, in the dental implantology field it is already known the need of forming a cavity in the gums and bone tissue and following to the extraction of a tooth from its seat, and with the aim of implanting a prosthesis or an implant for the supporting of a prosthetic tooth. With this aim, surgical tools such mechanical mills, sonic mills, laser or the like are typically provided, since the allow the making of bores of different shapes and sizes and according to the kind of prosthesis to be implanted in the bone and/or gums tissue of the patient.

According to the known methodology there are some drawbacks. A first drawback is due to the real difficulty of making the consequent boring which has its coaxiality maintained, i.e. when changing selectively tool in the evolution of the bore. This means that the boring has to be done by an extreme experienced surgeon which has to make the bore by hand with a continues gauging of the same. whereas the results are never reliable or guaranteed.

Another drawback consists in that when boring a bone tissue for the making of a seat for the implant, the bore obtained it is almost never shaped as the root of the tooth which was inside of the same before being extracted. This is due to the fact the surgical tool does not allows the making of bores with a concave or curved shape. This leads to the fact that the bore has always a substantially cylindrical shape, and only thanks to the extreme ability of the surgeon it is possible to make removal of small pieces of bone and/or gums tissue in order to obtain a predetermined shape necessary to house the implant.

### SUMMARY OF THE INVENTION

Object of the present invention is to provide a method and a system for the making of non cylindrical bores in organic tissues which solves the abovementioned drawbacks and allow to make bores in organic tissues for the housing of prosthesis having no cylindrical shapes and with the use of surgical tools of the state of the art, and guaranteeing the at the same time duplicating and maximum precision in the boring operation.

Another object of the present invention is to provide a method and a system for the making of non cylindrical bores in organic tissues which allows the making of subsequent bores with a perfect coaxiality.

Further object of the present invention, is to provide a method and a system for the making of non cylindrical bores in organic tissues which allows the boring operation by means of surgical tools of the state of the art, such as mechanical mills, sonic mills, or laser tools, and with an extreme ease, safety and reliability.

Therefore, the present invention provides a system and a method according to the annexed claims.

### BRIEF DESCRIPTION OF THE FIGURES

A detailed description of a preferred embodiment of the system and the method for the making of non cylindrical bores in organic tissues according to the present invention will be given hereinafter, and as a non limiting example, with reference to the annexed drawings, wherein:

FIGS. 1 to 4 show in different perspective views a first component of the system of the present invention;

FIGS. 5 to 8 show in different perspective views a second component of the system of the present invention;

FIGS. 9A, 9B and 9C show schematically the method for the making of non cylindrical bores according to the present invention;

FIGS. from 10A to 10F schematically show in a perspective views the steps of the method according to the present invention;

FIGS. from 11 to 14 show in perspective views a third component according to the system of the present invention;

FIGS. from 15A to 15B are perspective views of a different embodiment of the method for the making of non cylindrical bores according to the present invention; and

FIGS. from 16A a 16D are perspective views of the steps for the method of the present invention according to different embodiment thereof.

### DETAILED DESCRIPTION OF THE INVENTION

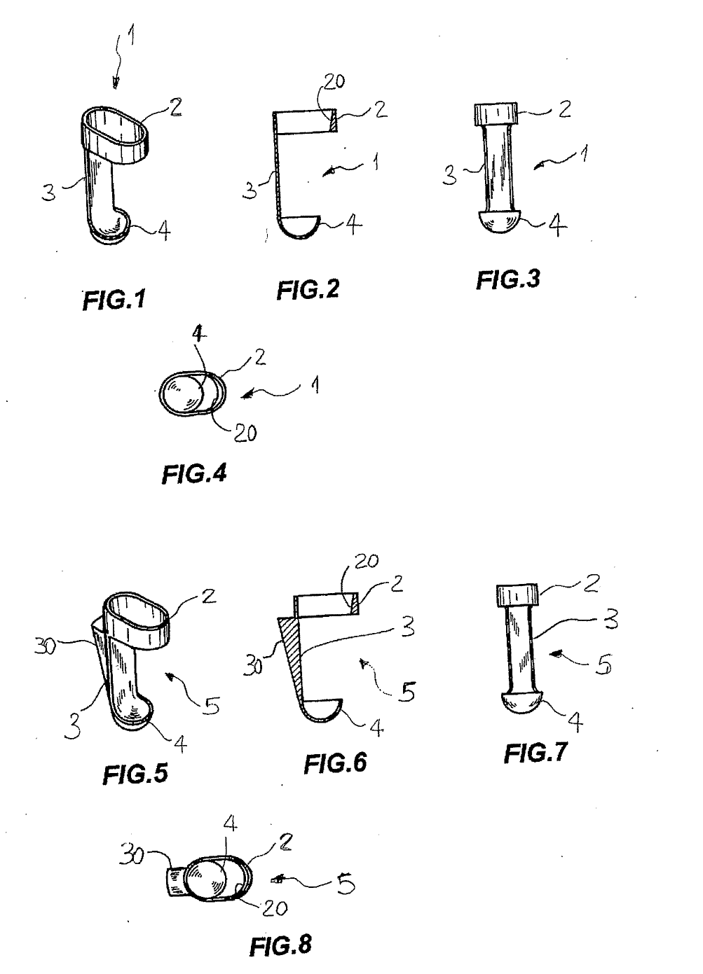

With reference now to FIGS. 1 to 4, they show in different perspective views a first component of the system for the making of non cylindrical bores of the present invention.

According to the present invention, the system provides a first template 1 which has a rigid body having an elongated shape, and a ring 2 substantially elliptical shaped or elongated “O” shaped, and which at a first end thereof is integrally connected to an elongated member 3 ending with a concave end 4. As will be better understood in the following, at the inner part of the ring 2 (FIG. 2) and at the opposite side of the projecting part 3 there is provided an inner edge 20 which is not parallel but is inclined with respect to the projecting part 3. The arrangement between edge 20, projecting part 3, and concave part 4, is such that the template 1 realise a seat which follows a conical pattern and for the guiding of a surgical tool such as a mechanical mill, a sonic mill, or a supersonic mill, or a laser surgical tool (not shown in the figures and better described in the following).

With reference to FIGS. from 5 to 8 it is shown therein with different perspective views a second component of the system for the making of non cylindrical bores of the present invention. For the sake of clarity, same parts will have same reference numerals.

According to the system of the present invention, a second component 5 analogous to said first component 1, the former consisting of a template 5 having an elongated rigid body and a ring 2, the ring 2 having a substantially elliptical shape or elongated “O”, as the ring of said template 1, said ring 2 having at a first end thereof an elongated projecting part 3 integrally connected thereto, the part 3 ending with a concave part 4.

At the outer edge of said template 5 the projecting part 3 has a part 30 which presents the same taper of the seat of the template 1. On the other hand, at the inner part of the ring 2 and opposite to the projecting part 3 (FIG. 6), an inclined edge 20 with respect to the projecting part 3 it is provided. As it will be better understood in the following, the arrangement between edge 20, projecting part 3, and concave part 4 is such that the template 5 realises a seat which follows the conical pattern for the guiding of a surgical tool (not shown in the figures and better illustrated in the following).

With reference to FIGS. from 9A to 9C the method for the making of non cylindrical bores with the system of the present invention it is shown. It is better to precise here that in the figures the making of a conical bore in a bone tissue such as a mandibular bone it is shown, but the method and the system of the present invention can be applied to any similar organic tissue.

According to the method of the present invention, for the implanting of a dental prosthesis following to an extraction, first a seat for the bone implant has to be realised. Therefore, at first a standard cylindrical boring in a bone tissue T has to be made with a suitable surgical tool such as a mechanical mill, or a sonic or supersonic mill, or laser tool (not shown in the figures).

Subsequently to the making of the cylindrical bore in the tissue T, the template 1 is inserted therein. It is better here specify that when the template 1 is inserted in the bore inside the tissue T the depth of the projecting part 3 of the template 1 equals-substantially to that of the bore. Therefore, in this condition the ring 2 projects out of the bore and lies onto the outer portion of the tissue and peripherally to the bore.

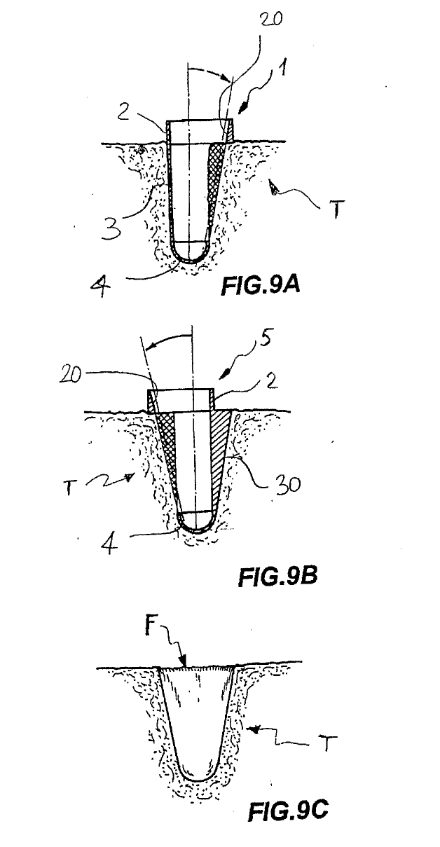

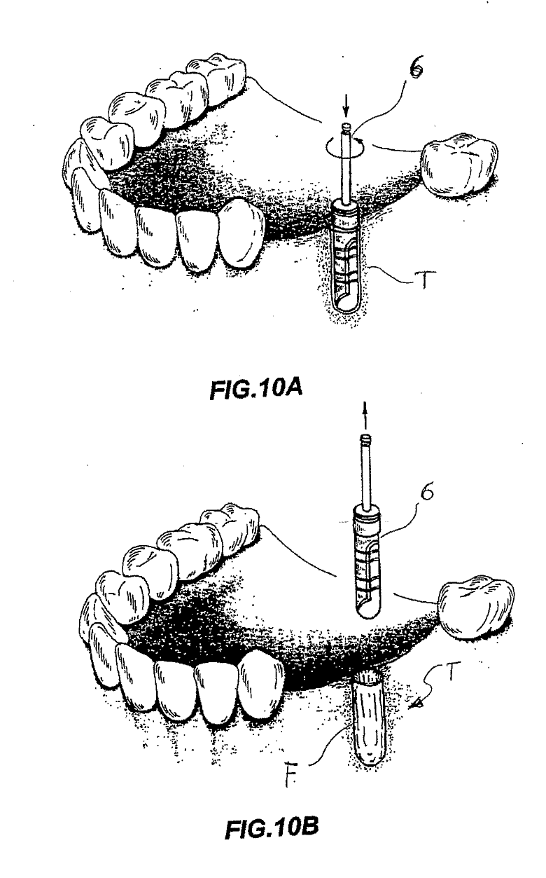

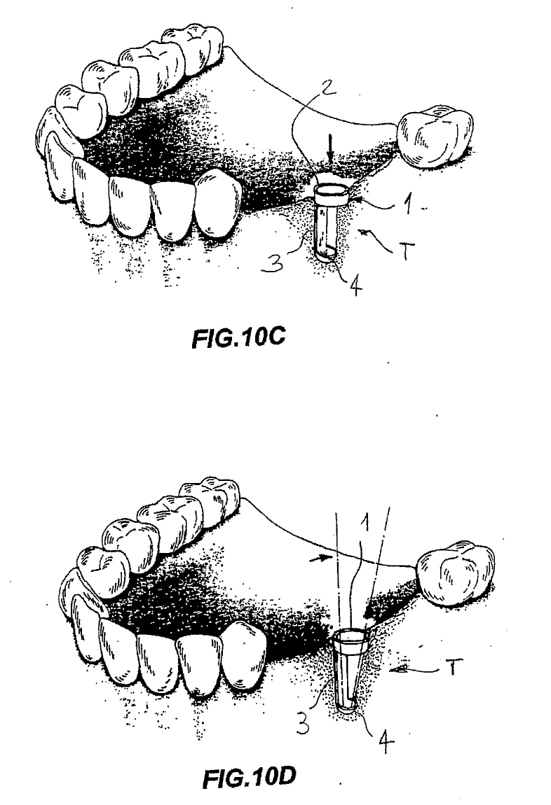

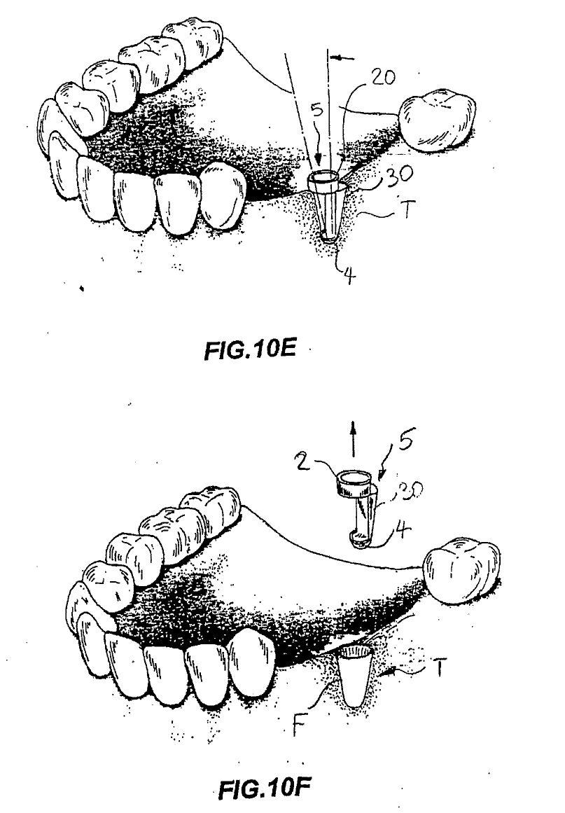

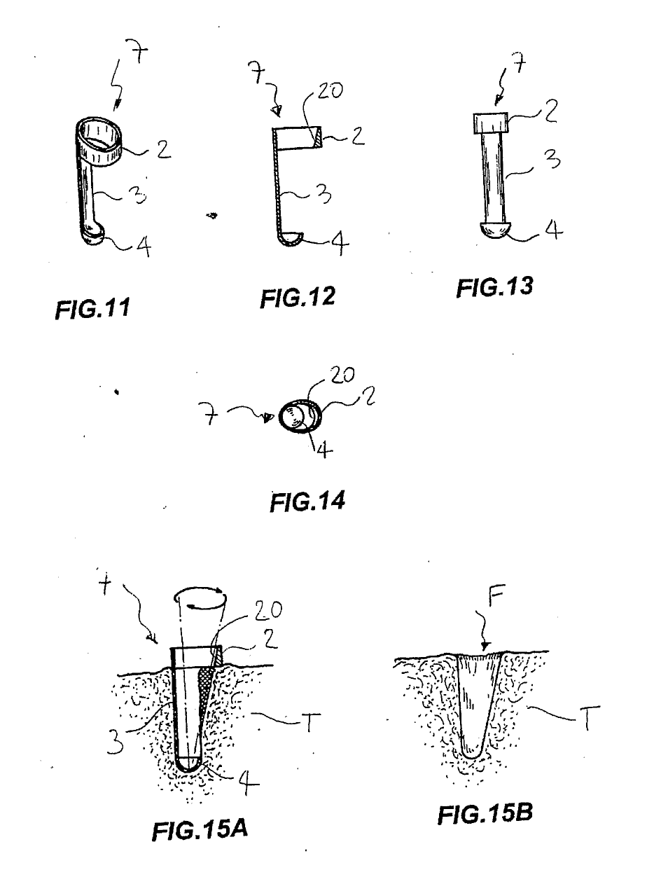

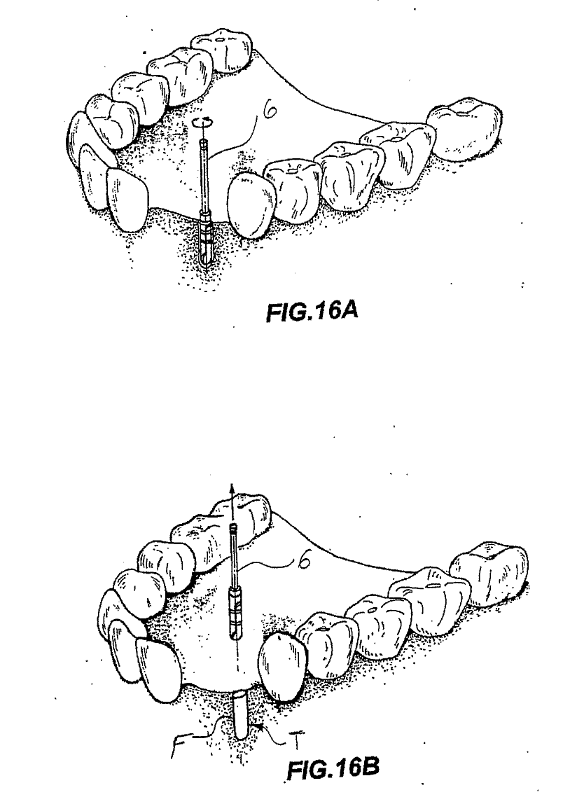

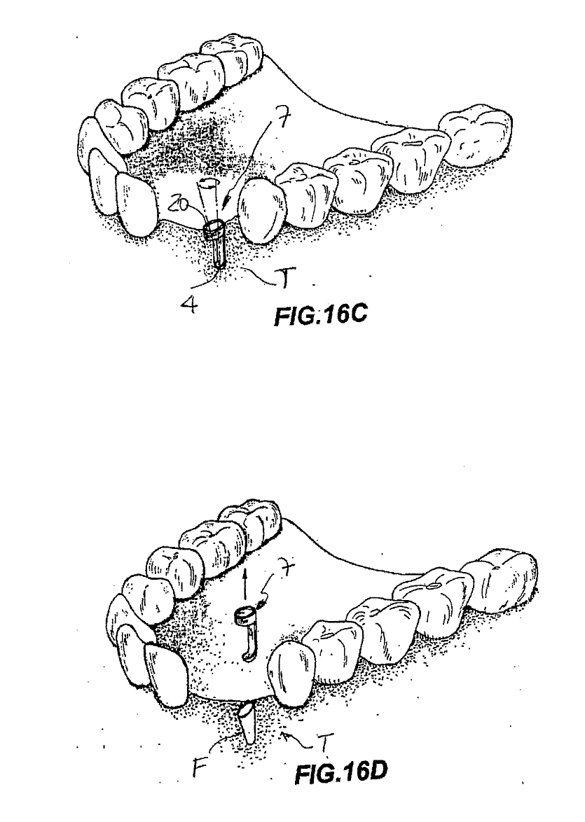

In this condition, it is possible to insert a surgical tool for the removal of tissue, i.e. a mechanical or sonic mill, or a laser tool (as the kind already used for this field), and up to reach the concave part 4 which coincides with the deepest part of the bore (the surgical tool it is not shown in the figures). By making a circular motion following the inner edge 20 of the ring 2, a removal of biological it is obtained which will constitute a seat or conical bore having the axis which follows the longitudinal axis of the template 1 (FIG. 9A).

Subsequently, after the tissue removal the template 1 is removed from the shaped bore and the template 5 is inserted therein following the shape of the bore which is analogous and coincides to the shape of the template 5. More precisely, the part 30 of the template 5 coincides with the conical wall of the bore obtained with the template 1. As per above, the template 5 has a length which equals the depth of the bore in the tissue T, and in this condition the ring member 2 projects from the bore and lies onto the peripheral part of the same.

Therefore, it is possible to insert a mill for the removal of organic material (of the same kind above described) inside the template 5 up to reach the concave part 4. Then, by removing tissue (by means of said surgical tool and not shown in the figures) and following the inner perimeter of the ring 2, a removal of organic material or boring having a conical shaped is obtained thanks to the inclination of the edge 20.

As can be noted in the FIG. 9C, after the removal of the template 5 from the tissue T a conical bore F it is obtained, the same reproducing substantially the shape of the implant to be inserted therein. It has to be noted here that when applying dental implants with a conical shape (substantially conical) this shape is the one who ensure the most reliability in the dental prosthesis performances when inserted in a bone tissue, thanks to the similarity to the shapes to those of the dental roots.

With reference now to FIGS. from 10A to 10F the method for the making of conical bores of the present invention and as above described, it is shown. More precisely, figures show the making of a conical bore for a dental implant mandibular corresponding to the 3rd or 4th molar, and wherein a starting boring with a mechanical mill is made.

As can be understood from the figures, first a bore in a bone tissue T is made by means of a state of the art mill 6, the mill 6 realises a cylindrical shaped bore F in the tissue T.

Therefore, after the cylindrical bore F has been made of a predetermined depth, the template 1 is inserted in the former. In this condition, with a suitable surgical tool for the removal of tissue, such as mechanical mill, sonic or supersonic mill, or a laser tool (not shown in the figures) it is possible realise a substantially conical bore by lying the tool at the bottom concave part 4 and peripheral following the inner edge 20 (FIG. 10D).

Then, the template 1 is removed for inserting in the shaped bore the second template 5. As can be noted in FIG. 10E, the template 5 lies the part 30 onto the inclined wall of the bore F. In this condition, the removal operation is repeated with the same tool and following the perimeter of the edge 20 of the ring 2. When the removal of organic tissue is finished, the template 5 is removed from the bore F leaving a substantially conical and symmetrical shaped bore. In this condition is possible to insert the implant according to the already known methodology, and advantageously guaranteeing top reliability and precision, impossible to the date.

With reference now to FIGS. 11 and 12, a third component of the system for the making of non cylindrical bores of the present invention is shown therein. As already shown up to now, same parts will have same reference numerals for the sake of clarity.

According to the system of the present invention, a third component, is provided the same consisting of a template 7 having a rigid elongated shaped body having a ring 2 substantially elliptical shaped or “O” elongated shaped. The ring 2 is integrally connected at an end thereof to an elongated projecting part 3 which ends with a concave part 4. At the inner part of the ring 2 (FIG. 12) and at the opposite side where the part 3 is connected to, an inclined edge 20 with respect to the part 3 is obtained. As will be better understood in the following, the shape of the ring member 2 of the template 7 allows the making of conical bores having shaped which are suitable for dental implants relating to front teeth of a person (i.e., single rooted teeth).

As above already described, also in this condition the arrangement between edge 20, projecting part 3, and concave part 4 realises a seat having a conical pattern for the guiding of a tissue removal tool (not shown in the figures and better illustrated in the following).

With reference now to FIGS. 15A and 15B, the method for the making of a conical bore with the template 7 it is schematically shown and according to the system of the present invention. Therefore, as above already described, first a cylindrical boring in a bone tissue T is made with a suitable state of the art tool, such as a mill or similar (not shown in the figures). Similarly, the depth of the template 7 substantially equals to that of the bore F in the tissue T.

Then, in the bore in the tissue T the template 7 is inserted, and the ring 2 projects from the bore, and lies onto the outer part of the tissue and peripheral to the bore F. In this condition, it is possible insert the surgical tissue removal tool as above described, and up to reach the concave part 4 which coincides with the deepest part of the bore. Per the removal of the tissue a circular motion is realised and following the perimeter of the inner edge 20 of the ring 2, and a removal of bone tissue is obtained and a conical seat or bore having the same axis of the longitudinal axis of the template 7 is obtained (FIG. 15A).

Subsequently, the template 7 is extracted from the bore, which shape reproduces substantially the shape of the implant to be inserted therein.

With reference now to FIGS. from 16A to 16D, the method for the making of conical bores with the template 7 and relevant to a mandibular bone tissue and a front dental implant (i.e., implants for single rooted teeth) it is shown.

As can be understood from the figures, first a bore in the bone tissue T is obtained by means of a tissue removal tool, such as a state of the art mill 6. As it is apparent, the mill 6 realises a bore F in the tissue T having a shape cylindrical shape. Then, after the cylindrical bore F of a predetermined depth is made, the template 7 is inserted therein.

In this condition, with a-state of the art suitable tissue removal tool, such as a mechanical mill, sonic mill, or laser tool (not shown in the figures) it is possible realise a substantially conical bore by lying the mill in the bottom concave part 4 and following the edge 20 (FIG. 16C).

Then, when the template 7 is removed the bore has a substantially conical shape and reproduces the shape of the tooth root. In this condition, is possible to apply an implant according to the methodology already known and advantageously guaranteeing top reliability and precision.

1. Template (1) for the making of non cylindrical bores in organic tissues or the like by means of a surgical tool,
characterised in that it comprises an annular member (2) substantially elliptically or “O” elongated shaped, and an elongated member (3) integrally connected at a first end thereof to said annular member (2), and at the opposite end of said elongated member (3) being shaped with a concave end part (4).

2. Template (1) according to claim 1, wherein said annular member (2) has a inner edge part (20) at the opposite side to the side where said elongated member (3) is integrally connected, said inner edge part (20) being inclined with respect to the longitudinal direction of said elongated member (3), the arrangement between said edge (20), said elongated member (3) and said concave end part (4) being such that said template (1) realises a seat for the guiding of a surgical tool, said seat having a substantially frustoconical pattern.

3. Template (5) for the making of non cylindrical bores in organic tissues or the like by means of a surgical tool,
characterised in that it comprises an annular member (2) having a substantially elliptical or “O” elongated shape, and an elongated member (3), said elongated member being integrally connected at a first end thereof with a respective side end of said annular member (2), and at the opposite end of said elongated member (3) being shaped with a concave end part (4), and in that said elongated member (3) has a projecting part (30) which projects from an outer side of said template (5), said projecting part (30) defining a conical surface part converging to said concave end part (4).

4. Template (5) according to claim 3, wherein said annular member (2) has an inner edge part (20) at the opposite side to the side where said elongated member (3) is integrally connected, said inner edge part (20) being inclined with respect to the longitudinal direction of said elongated member (3), the arrangement between said edge (20), said member elongated (3) and said concave end part (4) being such that the template (5) realises a seat for the housing of a surgical tool and which follows substantially frustoconical pattern.

5. Template (7) for the making of non cylindrical bores in organic tissues or the like by means of a surgical tool,
characterised in that it comprises an annular member (2) having a substantially elliptical or “O” elongated shaper and a elongated member (3), said elongated member (3) being integrally connected at a first end thereof with a respective side end of said annular member (2), and at the opposite end of said elongated member (3) being shaped with a concave end part (4).

6. Template (7) according to claim 5, wherein said annular member (2) has a inner edge part (20) at the opposite side to the side where said elongated member (3) is integrally connected, said inner edge part (20) being inclined with respect to the longitudinal direction of said elongated member (3), the arrangement between said edge (20), said elongated member (3) and said concave end part (4) being such that said template (1) realises a seat for the guiding of a surgical tool, said seat having a substantially frustoconical pattern.

7. Template (1;5;7) according to claim 1, wherein the length of said elongated member (3) is predetermined by the depth of the bore to be realised in said organic tissue or the like.

8. A system for the making of non cylindrical bores in organic tissues or the like by means of a surgical tool, characterised in that it comprises at least one of said templates (1;5;7) according to claim 1.

9. System for the making of non cylindrical bores in organic tissues or the like by means of a surgical tool according to claim 8, wherein said surgical tool comprises a mechanical mill.

10. System for the making of non cylindrical bores in organic tissues or the like by means of a surgical tool according to claim 8, wherein said surgical tool comprises a sonic mill.

11. System for the making of non cylindrical bores in organic tissues or the like by means of a surgical tool according to claim 8, wherein said surgical tool comprises a ultrasonic mill.

12. System for the making of non cylindrical bores in organic tissues or the like by means of a surgical tool according to claim 8, wherein said surgical tool comprises a laser.

13. A method for the making of non cylindrical bores in organic tissues or the like by means of a surgical tool, characterised in that it comprises the following steps:
boring a first cylindrical bore in a tissue (T) of a predetermined depth with a surgical tool (6);
inserting a template in said cylindrical bore, said template (1;5;7) having an elongated member (3) of a predetermined length with a concave end part (4), and a tool guiding annular member (2) with an inclined inner edge (20);
inserting a surgical tool inside said template (1;5;7) and up to reach said concave end part (4) which coincides with the deepest part of the bore;
making a circular motion with said tool following said inner edge (20) of said annular member (2), obtaining a removal of organic tissue with a frustoconical shaped bore; and
removing said template (1) from said frustoconical shaped bore.
